(19) United States
(12) Patent Application Publication
(10) Pub. No.: US 2014/0379069 A1
(43) Pub. Date: Dec. 25, 2014

(54) NEGATIVELY CHARGED VASCULAR STENT

(71) Applicant: HIPOKRAT, Ashkelon (IL)

(72) Inventors: Michael Fishman, Ashkelon (IL); Alexander Tsun, Ashdod (IL)

(21) Appl. No.: 14/375,435

(22) PCT Filed: Jan. 28, 2013
(86) PCT No.: PCT/IL2013/050074

§ 371 Date: Jul. 30, 2014

Related U.S. Application Data
(60) Provisional application No. 61/592,013, filed on Jan. 30, 2012.
(60) Provisional application No. 61/674,861, filed on Jul. 24, 2012.

(51) Int. Cl. A61F 2/90; C23C 14/34

(52) U.S. Cl. CPC A61F 2/90; C23C 14/3414; A61F 2240/001
USPC 623/1.15; 204/192.1

(57) ABSTRACT

An insertable into a human body article is coated with a coating of electret structure. The coating has an integral surface which blankets an entire surface of the article.

### FIELD OF THE INVENTION

The invention relates to medical devices for mitigating thrombosis risk and more specifically to a stent carrying a stable negative charge.

### BACKGROUND

A stent is an artificial ‘tube’ inserted into a natural passage/conduit in the body to prevent, or counteract, a disease-induced, localized flow constriction. The stents are inserted into narrowed coronary arteries to help keep them open after balloon angioplasty. The stent then allows the normal flow of blood and oxygen to the heart. Stents placed in narrowed carotid arteries (the vessels in the front of the neck that supply blood to the brain) appear useful in treating patients at elevated risk for stroke.

WO/1996/041589 discloses a device for implantation in a vessel or hollow organ lumen in a human or animal body, such as a self-expanding stent, a cava filter, an embolizing means or a supporting means, comprises a wire frame with a plurality of interconnected cells made of at least two separate wire sections which are intercrossing at cell junctions and form closed cells. At the cell junctions the wires are knot to form a geometrical locking of the cells so that the wire-shaped cell sides in respective cells are locked at the cell junctions when the wire frame is subjected to pressure acting radially inwards.

RU 2446775 discloses a stent includes a tubular element which has longitudinal axis. Tubular element contains first radial element and second radial element. First radial element contains elongated bridge with deflectable teeth. Bridge is characterized by upper and lower parts. Upper part narrows from first thickness to second thickness on first direction on circumference, approaching far edge of upper part. Lower part narrows from first thickness to second thickness in direction on circumference opposite to first direction, approaching far edge of lower part. Second radial element is located on circumference near first radial element and contains gearing means, which have configuration for sliding gearing of elongated bridge of first radial element and deflection of deflectable teeth, when teeth contact with gearing means in such a way that tubular element reaches extension in direction on circumference with reduced kickback. Gearing means narrow to second thickness in direction on circumference, approaching far edge of gearing means. In second version of stent implementation gearing means of second radial element include slot intended for sliding gearing of first radial element bridge and deflection of teeth, when elongated bridge of first radial element passes through slot, in such a way that tubular element obtains extension in direction on circumference with reduced kickback. Second radial element does not overlap itself when stent is in extended state. Gearing means narrow on circumference to second thickness, approaching far edge of gearing means in such a way, that first and second radial elements preserve mainly constant common thickness, when first radial element is geared by gearing means of second radial element and stent is in extended state.

U.S. Pat. No. 6,231,600 discloses a device such as a stent provided with a hybrid coating including a time released, restenosis inhibiting coating and a non-thrombogenic coating to prevent clotting on the device. One first coat or layer includes a polymer, a crosslinking agent, and pacitaxel, analogues, or derivatives thereof. The first coat preferably includes a polymer having Taxol admixed therein so as to be releasable over time. The first coat preferably includes a polyfunctional aziridine as the crosslinking agent. The second coat preferably includes heparin to inhibit clot formation on the device. The crosslinking agent can covalently bond to both the first coat polymer and the second coat heparin. A stent can be provided with a first coat including an aqueous dispersion or emulsion of a polymer and an excess of crosslinking agent. The first coating can be dried, leaving a water insoluble polymer coating. A second aqueous coating including a solution or dispersion of heparin can be applied over the first coating, the heparin becoming covalently bound to the crosslinking agent on the first coating surface. The resulting stent can inhibit restenosis while preventing blood clot formation on the stent.

RU 2380059 discloses a stent coating containing a polymer material with an active antiproliferative substance. The polymer material presents a copolymer of butyric and valeric acids, while the active antiproliferative substance is rubomycinum. The amount of the copolymer of butyric and valeric acids per one stent is 2-15 mg/stent, while rubomycinum composes a polymer layer in amount 0.002-0.025 mg/stent.

U.S. Patent Application 2008/0208315 disclosed a stent for coronary vessels, having a surface of multilayer immobilized structures, includes a stent body and a number of polyelectrolyte complex (PEC) layers stacking and being immobilized on the surface of the stent body, in which the PEC layer is formed of a polymer layer and an anticoagulant layer. The coronary stent is capable of effectively improving the hemocompatibility and longevity over a conventional stent using surface encapsulation of an anticoagulant layer for hemocompatibility improvement. Furthermore, the coronary stent can be use as a drug-eluting coronary stent, thus allowing for the time-releasing of drugs, and further preventing the thickening of vascular smooth muscle cells for causing vascular thrombosis.

It is known in the prior art that blood cells being components of thrombosis such as thrombocytes and leucocytes are negatively charged. U.S. Patent Application 2006/0106451 discloses an electronic anti-coagulation stent structure. The stent structure comprises a pair of coaxial metal stents having a layer of dielectric material between the stents. A battery is operatively connected preferably near or adjacent to the upstream end upon deployment of the stent. The positive battery terminal establishes an electrical connection to the outer metal stent and the negative terminal establishes an electrical connection to the inner metal stent and this exhibits a capacitor-like properties. The inner metal stent, being negatively charged, promotes a platelet repellent, anti-thrombotic effect.

U.S. Patent Application 2011/0196478 discloses devices and methods for lumen treatment. There is provided an endoprosthesis that includes an internal layer designed to provide a negative electric field directed endoluminally; an external layer designed to provide a positive electric field directed exoluminally; and one or more intermediate layers disposed between the internal layer and the external layer, wherein the negative electric field is due to a negative point charge between about −25 mV and about −250 mV, and wherein the positive electric field is due to a positive point charge between about +1 mV and about +30 mV.

Life span of the battery limits duration of the stent use. When exhausted, the battery should be replaced by means of a surgical procedure.

The vascular stents known in the art are characterized by substantive hydrophilicity. Thrombogenic blood particles freely penetrate into the gaps between stent elements or between the stent and vascular walls and aggregate into a new clot. The chances that surgically treated blood vessels will be re-stenosed are very good. Thus, there is a long-felt and unmet need to provide a hydrophobic vascular stent which is able to repel the aforesaid thrombogenic blood particles and prevents the surgically treated blood vessels from recurrent stenosis and sclerosis.

### SUMMARY OF THE INVENTION

It is hence one object of the invention to disclose an article insertable into a patient's body.

It is a core purpose of the invention to provide the article coated with a coating having an electret structure.

Another object of the invention is to disclose the article selected from the group consisting of a vascular stent, an artificial cardiac valve, a suture material, orthopedic article, applicator, hernia mesh, acupuncture needle including corporeal needle, otic needle, long-term needle, ball, plate, and any combination thereof. Another object of the invention is to disclose a vascular stent carrying a negative charge. The aforesaid stent comprises at least one metal wire member configured into a cylindrical celled frame.

A further object of the invention is to disclose the coating having an integral surface blanketing an entire surface of said wire member.

A further object of the invention is to disclose the coating which is tantalum pentoxide.

A further object of the invention is to disclose the tantalum pentoxide coating which is vacuum-plasma sputtering.

A further object of the invention is to disclose the coating is Polytetrafluoroethylene.

A further object of the invention is to disclose the vascular stent constructed of tubing cut into a mesh shape.

A further object of the invention is to disclose the vascular stent constructed of at least one wire into a mesh shape.

A further object of the invention is to disclose the stent in which a distance between each two neighboring wires of said stent is less 15 wire diameters such that said stent provides substantially uniform electrostatic field preventing blood cells from aggregation on the stent wires.

A further object of the invention is to disclose a method of manufacturing a vascular stent carrying a negative charge and insertable into a patient's body. The aforesaid method comprises the steps of: (a) providing an article work piece; and (b) coating said wire members with a coating carrying a negative charge; and

It is another core purpose of the invention to provide the step of coating article work piece further comprises creating an electret structure of said work piece.

A further object of the invention is to disclose the step of coating said wire members further comprising creating an integral surface blanketing an entire surface of said wire member.

A further object of the invention is to disclose the step of coating said wire members further comprising sputtering tantalum pentoxide.

A further object of the invention is to disclose the step of sputtering tantalum pentoxide which is performed by means of vacuum-plasma sputtering.

### BRIEF DESCRIPTION OF THE DRAWINGS

In order to understand the invention and to see how it may be implemented in practice, a plurality of embodiments is adapted to now be described, by way of non-limiting example only, with reference to the accompanying drawings, in which

FIGS. 1a and 1b are general views of a wire wound stents;

FIG. 1c is a general view of a stent braided of a wire;

FIG. 1d is a general view of a stent cut of a tube by a laser;

FIG. 2a is an exemplar view of a mesh structure which is cut by a laser;

FIG. 2b is an exemplar view of a mesh structure formed of wavelike wires;

FIG. 2c is an exemplar view of a mesh structure formed of interlaced wires;

FIG. 3a is an illustration specifying aggregation of blood particles into a clot; and

FIG. 3b is an illustration specifying prevention of blood particles from aggregation into a clot.

### DETAILED DESCRIPTION OF THE INVENTION

The following description is provided, so as to enable any person skilled in the art to make use of said invention and sets forth the best modes contemplated by the inventor of carrying out this invention. Various modifications, however, are adapted to remain apparent to those skilled in the art, since the generic principles of the present invention have been defined specifically to provide a vascular stent carrying a negative charge and a method of manufacturing the same.

The core objective of the present invention is to reduce the risk of recurrent thrombosis. The vascular stent of the present invention is characterized high biological compatibility including hemocompatibility and temporally stable anti-coagulating and anti-clotting properties implantable into coronary or other arteries to restore patency thereof. A vascular stent constituting a cylindrical celled frame made of at least one wire member is coated with a coating having an electret structure. The aforesaid coating carries negative surface charge. It should be emphasized that the electret coating has an integral surface blanketing an entire surface of the stent. In other words, there is a negative potential on entire surface of the stent. Hence, external stent surfaces directed to the vessel wall and internal stent surfaces directed to an internal space thereof carry a negative electric charge. According to one embodiment of the present invention, the electret structure is formed by means of coating the stent with tantalum pentoxide (Ta₂O₅). According to an alternative embodiment of the present invention, the electret coating is made of polytetrafluoroethylene (Teflon®).

Negative charge carried by the vascular stent coated with electret films prevents blood plates such as thrombocytes or leucocytes from aggregation at a stent mounted within a blood vessel. It is known that the blood cells to be aggregated at the stent are negatively charged. Electronegativity of the stent surface results in prevention of blood cell aggregation both at the stent per se and the vascular wall-adjacent area due to repelling Coulomb forces. Thus, the negative surface charge generates electric static field shielding areas potentially prone to blood cell aggregation. The vascular stent carrying the electret coating decreases risk of restenosis. Life quality and longevity of the patients stented with temporally stable anti-coagulating and anti-clotting properties are improved in comparison with the unstented patient. Doses of anti-coagulating and anti-clotting medicine are cut as well. In accordance with common practice in the relevant field of technology, the stent is expanded due to plastic deformation when mounted within the blood vessel, for example, by means of an inflatable balloon. At least one wire member is configured into a cylindrical celled structure. The aforesaid wire member is coated with tantalum pentoxide. In accordance with one embodiment of the present invention, the tantalum pentoxide coating is made by means of vacuum-plasma sputtering.

In accordance with another embodiment of the present invention, the wire member is coated with Polytetrafluoroethylene.

The stents provided with tantalum coating are characterized by higher reagent resistance in comparison with the gold coated stents. Tantalum pentoxide is dielectric and characterized by high mechanical properties. Tantalum pentoxide is chemically inert and biologically compatible. Thus, the stent provided with tantalum pentoxide coating is safely implantable into blood vessel and may reside in situ for a long time without expected side effects such as toxicity from stent degradation products and repeat thrombosis at the stent.

The stent with tantalum oxide coating having electret structure is characterized by:
1. Tantalum oxide coating which is coated by means of vacuum-plasma sputtering has improved adhesion to a metallic surface due to the high-energy coating process providing covalent and ionic bondings between a material to be coated and plasma-spattered coating.
2. Negative charge of the electret film can be temporally stable for a predetermined period of time. It is known in the art that the time characteristic can be varied by means of changes in the technological process.
3. It is very important that the stent with tantalum oxide coating can sterilized at high temperature.
4. The tantalum pentoxide coating is characterized by essential radiopacity. No additional efforts should be made for X-ray reliable detectability of the stent coated with tantalum pentoxide.

In accordance with the present invention, at least one wire member is coated with tantalum pentoxide, and then the obtained coated wire member(s) is configured into a stent expandable due to plastic deformation. It should be emphasized that local discontinuances in integrality of tantalum pentoxide coating result in local occurrence of positive electric potentials. As mentioned above, the blood cells which are aggregated in thrombosis are negatively charged. Thus, discontinuances in coating integrality will be centers of clottage/thrombosis. The coating process of the entire stent is very problematic. Specifically, adhesion of the coating in intertangling areas is much lower than the adhesion at linear areas. In accordance with the proposed innovation, wire member is configured into the celled structure after coating with tantalum pentoxide. In the present technical solution, adhesion value does not depend on a position on the stent and has a constant value in any location on the stent. Thus, there are no discontinuances in the coating integrality, and positive potential does not occur in any location. The entire stent is negatively charged and prevents any clottage/thrombosis thereon.

Technological improvement required for practical realization of the present invention is minor because a metal wire used for producing vascular stents should be replaced with a wire coated with an electret coating. Thus, there is no need to change mass production machines producing the vascular stents.

The negatively charged stent can be made of a number of wire members which are coated with tantalum pentoxide before. In this case, impermeability of the coating is not broken, and the positive charge does not occur at the stent surface, because coating integrality of each wire member is kept during a process of stent fabrication.

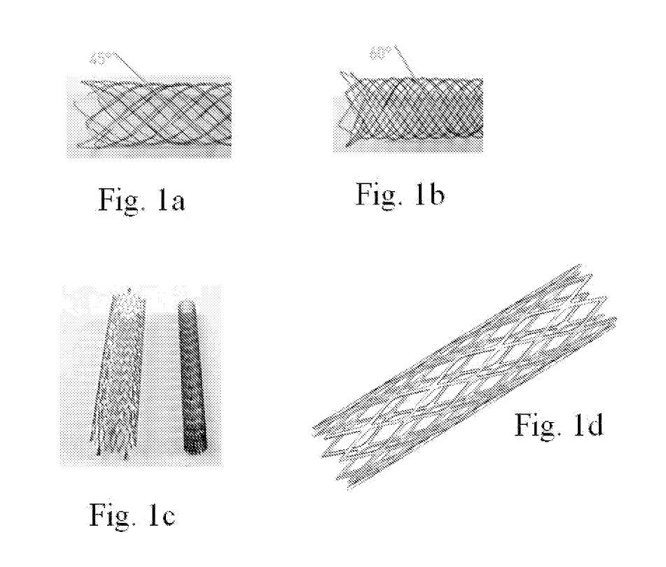

Reference is now made to FIGS. 1a to 1d illustrating general views of commercially available stents. Specifically, FIG. 1a and FIG. 1b show wire wound stents. FIGS. 1c and FIG. 1d illustrate a braided stent and a mesh-like stent laser cut of a tube, respectively.

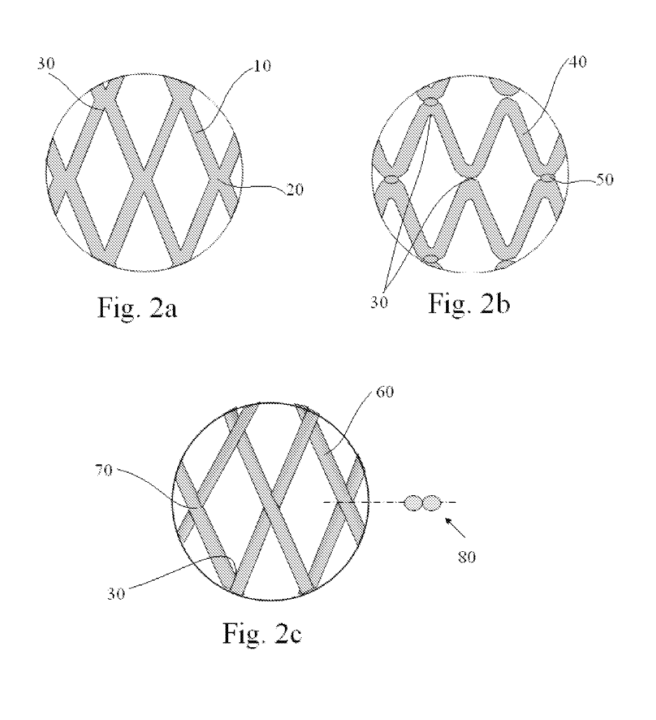

Reference is now made to FIG. 2a, presenting an exemplar mesh-like structure made by means of laser cutting. Numerals 10 and 20 refer to a wire element and a node, respectively. Experiments proved that locations 30 and similar having the mesh elements 10 meeting together at the node 20 at an acute angle serve as points of clot formation. Reference is now made to FIG. 2b, showing an exemplar mesh-like structure made of wavelike wire elements 40 which are secured to each other by means of spot welding 50. As seen in the FIG. 2b, the locations 30 can be characterized as dangerous in terms of clot formation.

Reference is now made to FIG. 2c, presenting an exemplar mesh constituting an interlaced wire structure. Wires 60 are interlaced in locations 70 which are points of clot formation. A numeral 80 refers to a cross-sectional view of the interlaced wires 60.

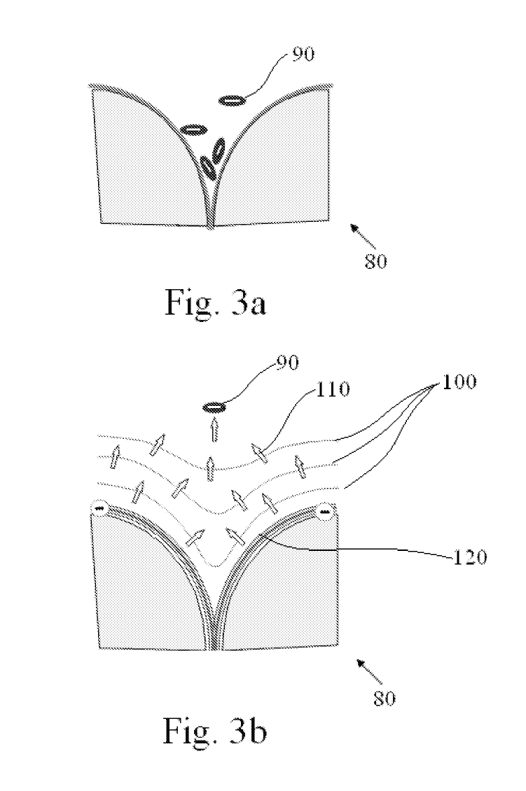

Reference is now made to FIG. 3a which is an illustration specifying aggregation of blood particles into a clot. Specifically, thrombocytes 90 aggregate in proximity of interlaced wires and form a clot.

Reference is now made to FIG. 3b, showing claimed prevention of the thrombocytes from aggregation in proximity of dangerous locations. As known in the art, the thrombocytes carry negative electric charge. An electret coating also carries a permanent negative charge and repels the thrombocytes from the dangerous locations and hence prevents the thrombocytes from aggregation in proximity of the stent. Equipotential lines 100 characterize electric field intensity in proximity of the interlaced wires. A direction of a repelling force acting onto the thrombocytes 90 is indicated by a numeral 110.

It should be emphasized that the method of manufacturing the claimed article comprises a step of expanding the article to be coated to prevent shaded areas to provide an integral surface blanketing an entire surface of said wire member. The expanded stent is mounted on a massive base plate providing effective heat sinking to prevent the stent to be coated from overheating during the coating process.

In accordance with a preferred embodiment of the present invention, in any cross section of the vascular stent, a distance between two neighboring wires is less 15 wire diameters. The described stent arrangement provides substantially uniform electrostatic field preventing blood cells from aggregation on the stent wires.

Polymer articles having electret properties due to modifying a surface thereof by a corona discharge or other effects are in the scope of the present invention.

In accordance with the present invention, an article insertable into a patient's body,

A core feature is to provide the article coated with a coating having an electret structure.

In accordance with another the present invention, the article is selected from the group consisting of a vascular stent, an artificial cardiac valve, a suture material, orthopedic article, applicator, hernia mesh, an acupuncture needle including corporeal needle, otic needle, long-term needle, ball, plate, and any combination thereof.

In accordance with a further the present invention, a vascular stent carrying a surface negative charge is disclosed. The aforesaid stent comprises at least one metal wire member configured into a cylindrical celled frame.

Another core feature is to provide the wire member coated with a coating having an electret structure.

In accordance with further the present invention, the coating has an integral surface blanketing an entire surface of said wire member.

In accordance with further the present invention, the vascular stent is constructed of tubing cut into a mesh shape.

In accordance with further the present invention, the vascular stent is constructed of at least one wire into a mesh shape.

In accordance with further the present invention, the coating is tantalum pentoxide.

In accordance with a further the present invention, the tantalum pentoxide coating is vacuum-plasma sputtering.

A further object of the invention is to disclose the said coating is Polytetrafluoroethylene.

In accordance with a further the present invention, a method of manufacturing an article carrying a negative charge and insertable into a patient's body is disclosed. The aforesaid method comprises the steps of: (a) providing an article work piece; and (b) coating said work piece with a coating carrying a negative charge.

It is another core feature to provide the step of coating said work piece further comprising creating an electret structure of said work piece.

In accordance with a further the present invention, the step of coating said wire members further comprises creating an integral surface blanketing an entire surface of said wire member.

In accordance with a further the present invention, the step of coating said wire members further comprises sputtering tantalum pentoxide.

In accordance with a further the present invention, the step of sputtering tantalum pentoxide is performed by means of vacuum-plasma sputtering.

1-18. (canceled)

19. An article insertable into a patient's body, said article coated with a coating having an electret structure; wherein said electret coating comprises tantalum pentoxide.

20. The article according to claim 19 selected from the group consisting of a vascular stent, an artificial cardiac valve, a suture material, orthopedic article applicator, hernia mesh, an acupuncture needle including corporeal needle, otic needle, long-term needle, ball, plate and any combination thereof.

21. The article according to claim 19, wherein said coating has an integral surface blanketing an entire surface of said wire member.

22. The article according to claim 21, wherein said tantalum pentoxide coating is made by a vacuum-plasma sputtering method.

23. The article according to claim 19, wherein said coating comprises polytetrafluoroethyléne.

24. The article according to claim 20, wherein said vascular stent is constructed of tubing cut into a mesh shape.

25. The article according to claim 20, wherein said vascular stent is constructed of at least one wire configured into a mesh shape.

26. The article according to claim 24, wherein a distance between each two neighboring mesh elements of said stent is less 15 a caliper of said elements such that said stent provides substantially uniform electrostatic field preventing blood cells from aggregation on said mesh elements.

27. The article according to claim 25 being expandable such that mesh elements are exposed to electret coating without shading.

28. The article according to claim 24 being expandable such that mesh elements are exposed to electret coating without shading.

29. The article according to claim 25 being expandable such that mesh elements are exposed to electret coating without shading.

30. A method of manufacturing an article carrying a negative charge and insertable into a patient's body; said method comprising the steps of:
a. providing an article work piece;
b electretyoating said work piece;
Wherein said step of el ectretcoáting said work piece further comprises coating said work piece with tantalum pentoxide in an expanded configuration of said work piece.

31. The method according to claim 30 wherein said work piece is mounted on a massive base plate providing effective heat sinking.

32. The method according to claim 30, wherein said article is selected from the group consisting of a vascular stent, an artificial cardiac valve, a suture material, orthopedic article, applicator, hernia mesh, an acupuncture needle including corporeal needle, otic needle, long-term needle, ball, plate, and any combination thereof.

33. The method according to claim 30, wherein said article work piece is selected from the group consisting of a wire stent and a mesh stent cut of a metal tubing.

34. The method according to claim 30, wherein said step of coating said wire members further comprises creating an integral surface blanketing an entire surface of said wire member.

35. The method according to claim 30, wherein said step of sputtering tantalum pentoxide is performed by means of vacuum-plasma sputtering.

36. The method according to claim 30, wherein said step of coating said wire members further comprises coating with Polyteträfluoroethylene.
